(19) United States
(12) Patent Application Publication
(10) Pub. No.: US 2023/0233440 A1
(43) Pub. Date: Jul. 27, 2023

(54) SKIN CARE SERUM CONTAINING SODIUM HYALURONATE

(71) Applicant: The Procter & Gamble Company, Cincinnati, OH (US)

(72) Inventors: Joseph Harry Jansen, Harrison, OH (US); Monica Salguero Marlow, Mason, OH (US); Cindy Lucille Lewis, West Chester Township, OH (US); Renan Preda de Oliveira, West Chester, OH (US)

(21) Appl. No.: 17/582,237

(22) Filed: Jan. 24, 2022

(51) Int. Cl. A61K 8/73; A61K 8/04; A61K 8/891; A61K 8/81; A61K 8/31; A61K 8/365; A61K 8/34; A61K 8/67; A61Q 19/00

(52) U.S. Cl. CPC A61K 8/735; A61K 8/04; A61K 8/891; A61K 8/8158; A61K 8/31; A61K 8/365; A61K 8/345; A61K 8/675; A61Q 19/00; A61K 2800/48; A61K 2800/87; A61K 2800/30

(57) ABSTRACT

A physically stable skin care serum emulsion composition with a continuous phase and a dispersed phase. The continuous phase can include from about 0.5% to about 1.5% sodium hyaluronate with a weight average molecular weight from about 500,000 to about 1,000,000 Da according to the European Pharmacopoeia Method. The continuous phase can also include a thickening agent that includes polyacrylamide polymers and copolymers. The dispersed phase can include silicone. The composition can have an average viscosity from about 4,000 cP to about 25,000 cP and can be stored and dispense via a dropper package.

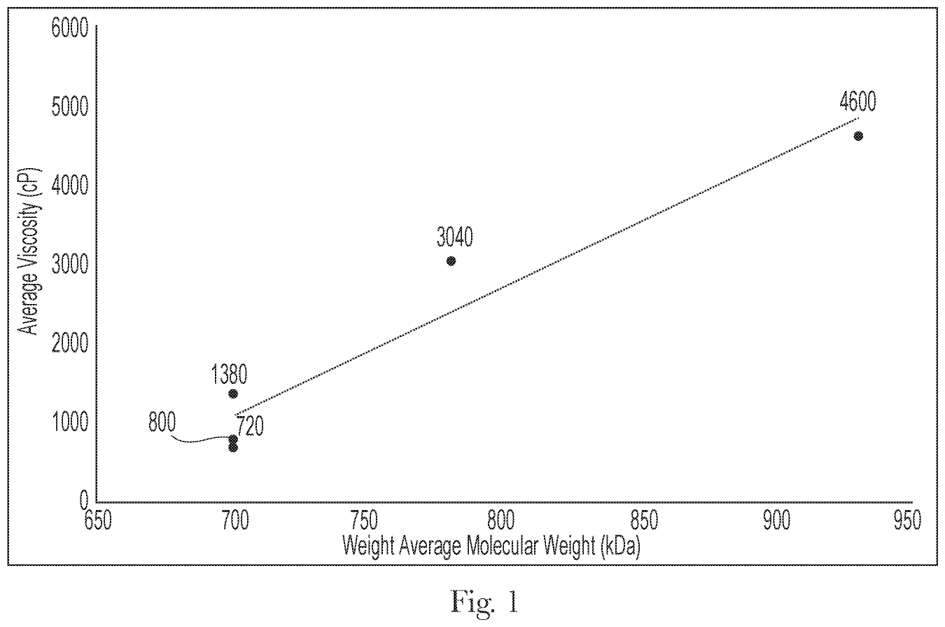

### FIELD OF THE INVENTION

The present invention is generally directed towards skin care compositions that contain hyaluronic acid and a thickening agent, in particular a physically stable skin care emulsion composition that contains greater than 0.5% hyaluronic acid with a weight average molecular weight of from about 200,000 Da to about 1.5 million Da, greater than or equal to 0.5% thickening agent, and an average viscosity of from about 3000 cP (2 Pa*s) to about 25,000 cP (25 Pa*s).

### BACKGROUND OF THE INVENTION

Many consumers want skin care products that contain sodium hyaluronate, particularly a relatively high level of sodium hyaluronate (e.g., ≥0.5%, ≥0.7%, ≥0.9%, or ≥1%). Sodium hyaluronate can increase skin hydration and reduce the appearance of fine lines and wrinkles.

Some consumers want sodium hyaluronate in a serum, which is a thin-viscosity topical product, because they believe this product form can be especially effective at quickly delivering on-skin results. Further, serums generally feel light enough that they can be used under a facial moisturizer, other facial treatments, or makeup.

Since serums can have a high concentration of active ingredients, only a few drops are generally needed to deliver results to the full face and/or neck and therefore many serums are dispensed from a dropper package. This package also allows consumers to customize their skin care regimen by adding droplets to another product. Further, the attractive dropper package can suggest effectiveness, as droppers were traditionally used in the pharmaceutical industry, while also connotating luxury and fashion.

However, it was found that a relatively high level of sodium hyaluronate unexpectedly builds viscosity when added to a traditional serum chassis, which, when combined with one or more traditional polymeric thickening agents, can make the formula too viscous to dispense with a dropper and/or easily distribute across a user's face. One way to decrease the viscosity is to eliminate ingredients that are known to build viscosity (e.g., thickening agents). However, the serum needs sufficient viscosity for physical stability (i.e., no phase separation) throughout the shelf life of the product.

Therefore, there is a need for a physically stable serum that contains a relatively high level of sodium hyaluronate that is compatible with a dropper dispenser.

### SUMMARY OF THE INVENTION

A physically stable skin care serum composition comprising: (a) a continuous phase comprising: (i) from about 0.5% to about 1.5% sodium hyaluronate comprising a weight average molecular weight from about 500,000 to about 1,000,000 Da; (ii) greater than 0.7% thickening agent; wherein the thickening agent is a polyacrylamide polymers and copolymers; (iii) water; (b) a dispersed phase comprising silicone; wherein the composition comprises a viscosity from about 4,000 cP to 25,000 cP.

A physically stable skin care serum composition comprising: (a) from about 0.5% to about 1.5% sodium hyaluronate comprising a weight average molecular weight from about 700,000 to about 1,000,000 Da; (b) greater than 1% thickening agent; wherein the thickening agent is polyacrylamide polymers and copolymers; (c) water; wherein the composition comprises a viscosity from about 4,000 cP to 25,000 cP.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

The Figure shows the impact on viscosity of a simple system when adjusting the weight average molecular weight of 1% sodium hyaluronate.

### DETAILED DESCRIPTION OF THE INVENTION

Hyaluronic acid is a biological polymer that occurs naturally in the skin and is the natural mucopolysaccharide formed by bonding N-acetyl-O-glucosamine with glucuronic acid. Sodium hyaluronate is the sodium salt of hyaluronic acid, and it can be used in skin care products.

Many consumers want skin care compositions that contain sodium hyaluronate, because it can regulate water within skin's surface cells and keep skin hydrated throughout the day. The mechanism by which hyaluronic acid and salts thereof hydrate skin can also help reduce the appearance of fine lines and wrinkles.

Many current marketed skin care compositions include relatively low levels of sodium hyaluronate. Some consumers may want skin care compositions that have a relatively high level (e.g., ≥0.5%, ≥0.7%, ≥0.9%, or ≥1%) of sodium hyaluronate, hoping to achieve even more moisture and/or anti-aging benefits. Many consumers want to apply skin care compositions with sodium hyaluronate in a serum, which is a thin-viscosity topical emulsion product that can be applied by a dropper.

However, it can be difficult to formulate a serum that has (1) low enough viscosity, so it is easy to dispense with a dropper and spread across the user's face and/or neck; (2) hydrophobic emollients, like silicone, that increase consumer delight during usage; and (3) physical stability.

It was discovered that ˜1% sodium hyaluronate unexpectedly built viscosity in compositions. It was also surprisingly found that the amount of viscosity built was correlated with the weight average molecular weight (Mw) of the sodium hyaluronate.

The Figure shows the impact of the weight average molecular weight of ˜1% sodium hyaluronate in a simple system (that contained 7% glycerin, 1% sodium hyaluronate, and the balance water) on average viscosity. The Figure shows that as the Mw of the sodium hyaluronate increases, the viscosity increases.

The weight average molecular weights were provided in a certificate of analysis by the supplier. The average viscosity was determined according to the following method: the viscosities for the simple system were measured after holding the sample for at least 24 hours at about 25° C. The viscosities of the examples are measured by a DV2T™-RV viscometer with RV3 spindle rotated at 5 rpm (available from Brookfield Engineering Laboratories, Middleboro, Mass.) at 25° C. The spindle is fully submerged, 2.5-3.0 cm below the composition's surface. The spindle is rotated for 30 s before starting data collection, then one data point is collected every 10 seconds for 5 readings, average (mean) the readings to determine the average viscosity.

Thus, simply adding a relatively high level of sodium hyaluronate to a serum formulation, especially a formulation that includes one or more thickening agents, could result in a product that had an unexpectedly high viscosity. Traditionally, product viscosity can be decreased by removing thickening agents in the formula. However, when thickening agents are removed from a serum it can become physically unstable, which can cause the sodium hyaluronate, emollients, and other skin care actives to fall out of the serum emulsion, which can appear as different layers in the serum product.

Next, formulations, as described in Table 1, below, were made to evaluate the product stability and viscosity at different levels of thickening agent (Polyacrylamide & C13-14 Isoparaffin & Laureth-7 commercially available as Sepigel™ 305 from Seppic® Corporation) in combination with 1% sodium hyaluronate with different weight average molecular weights. The results are summarized in Table 2, below. The average viscosity and stability were both determined after the compositions were stored for two weeks at 60° C.

TABLE 1

| | Material | Example (wt. %) |
|---|---|---|
| Water | Water | QS |
| Phase | Sodium Hyaluronate¹ | 1 |
| | Polyacrylamide and | 0.9-1.8 |
| | isoparaffin and laureth-7² | (as indicated in Table 2) |
| | Glycerin | 7 |
| | Niacinamide and other | 2.853 |
| | skin care actives | |
| | Chelant | 0.025 |
| | Antioxidant | 0.15 |
| | Preservative | 0.25 |
| | Emulsifier | 0.1 |
| Oil | 5 cst Dimethicone | 4 |
| Phase | Dimethicone (and) | 2 |
| | Dimethiconol³ | |
| | Emulsifiers | 0.4 |

¹The weight average molecular weight was 511,000 Da, 615,000 Da, 855,000 Da, or 920,000 Da, as indicated in Table 2, below
²Sepigel ™ 305 from Seppic ® Corporation
³DC1503 from Dow Coming ®

The average viscosity was determined by the Viscosity Test Method, described herein.

The weight average molecular weights of the sodium hyaluronate were provided in a certificate of analysis by the supplier.

The example was physically stable if by visual detection there is no phase separation. As used herein, “visual detection” means that a human viewer can visually discern if the product had separated into 2 distinct layers in a 2 oz clear glass jar with the unaided eye (excepting standard corrective lenses adapted to compensate for near-sightedness, farsightedness, or stigmatism, or other corrected vision) in lighting at least equal to the illumination of a standard 100-watt incandescent white light bulb at 30 cm.

TABLE 2

| | Mw of Sodium Hyaluronate | | | | | | | |
|---|---|---|---|---|---|---|---|---|
| Sepigel ™ | 511,000 Da | | 615,000 Da | | 855,000 Da | | 920,000 Da | |
| 305 | Viscosity | Physical | Viscosity | Physical | Viscosity | Physical | Viscosity | Physical |
| (wt. %) | (cP) | Stability | (cP) | Stability | (cP) | Stability | (cP) | Stability |
| 0.9 | 2000 | No | 3000 | No | 4000 | Yes | 3000 | Yes |
| 1.2 | 3000 | No | 3000 | No | 5000 | Yes | 5000 | Yes |
| 1.5 | — | | — | | 7000 | Yes | 6000 | Yes |
| 1.6 | 4000 | No | 5000 | Yes | — | | — | |
| 1.8 | 4000 | Yes | 6000 | Yes | 8000 | Yes | 9000 | Yes |

Table 2 shows that the average product viscosity increases as the concentration of Sepigel™ 305 increased, as well as when the Mw of the sodium hyaluronate increased. Also, when higher Mw sodium hyaluronate was used, the composition could have less thickening agent. For instance, in the compositions with the sodium hyaluronate that had an average Mw of 855,000 Da and 920,000 Da the composition was stable with 0.9% and 1.2% Sepigel™ 305 thickening agent. However, in the compositions with the sodium hyaluronate that had an average Mw of 511,000 Da and 615,000 Da they were both unstable with 0.9% and 1.2% Sepigel™ 305 thickening agent. These compositions were stable when 1.8% of thickening agent was added.

Next, formulations were made to test the functionality of the dropper package. To be functional, the dropper package needs to dispense enough product without too much effort. A formula was tested according to the formula in Table 1 with 1.8% thickening agent (Polyacrylamide & C13-14 Isoparaffin & Laureth-7 commercially available as Sepigel™ 305 from Seppic® Corporation) and 1% sodium hyaluronate with a Mw of with a 920,000 Da (hereinafter “High Viscosity Example”). The formula was put in the same dropper package as Olay® Regenerist® Retinol24® Night Facial Serum (“Retinol24®”). The weights of each dose from the dropper are compared to the weights of the dose from the dropper from Retinol24®, which is a successful in-market serum product with an ease of operation and dose that is acceptable to consumers. A dose was taken by squeezing the flexible top on the dropper to squeeze the air out, putting the tip into the serum, and then stop squeezing the top and the low pressure inside the dropper sucks up the liquid. The weight of the dose was then measured. The results are summarized in Table 3, below.

TABLE 3

| | Weight (g) | | | | |
|---|---|---|---|---|---|
| | Use 1 | Use 2 | Use 3 | Use 4 | Use 5 |
| Retinol24 ® | 0.43 | 0.47 | 0.48 | 0.52 | 0.58 |
| High Viscosity Example | 0.40 | 0.41 | 0.45 | 0.46 | 0.50 |

The product was considered consumer acceptable if the quantity of the dose from the dropper was within ±15% of the droplet size of the Retinol24® serum. The doses from the High Viscosity Example were within ±15% of the doses of the Retinol24® serum and it was easy to dispense. Therefore, the High Viscosity Example is consumer acceptable and the other physically stable Examples in Table 2 are also expected to be consumer acceptable because they are lower in viscosity than the High Viscosity Example.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

### Definitions

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein. In a specific example, an effective amount of a vitamin B₃ compound is an amount sufficient to improve the health and/or appearance of psoriatic skin during a treatment period. In some instances, an effective amount may be demonstrated using ex vivo and/or in vitro methods.

“Improve the appearance of” means providing a measurable, desirable change or benefit in skin appearance, which may be quantified, for example, by a decrease in redness, inflammation, and/or plaque scales.

“Safe and effective amount” means an effective amount of an ingredient that is low enough to avoid serious side effects (within the scope of sound medical judgment).

“Skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Treatment period,” as used herein, means the length of time and/or frequency that a material or composition is applied to a target skin surface.

### Composition

The skin care composition can be a serum with a viscosity that is sufficient for physical stability and is also thin enough so it can be dispensed by a dropper and easily spread across a user's face and/or neck. The average viscosity can be greater than 3000 cP (3 Pa*s), alternatively greater than 4000 cP (4 Pa*s), alternatively greater than 4500 cP (4.5 Pa*s), or alternatively greater than 5000 cP (5 Pa*s). The average viscosity can be less than 25,000 cP (25 Pa*s), alternatively less than 20,000 cP (20 Pa*s), alternatively less than 18,000 cP (1.8 Pa*s), alternatively less than 15,000 (15 Pa*s), alternatively less than 13,000 (13 Pa*s), or alternatively less than 10,000 (10 Pa*s). The average viscosity can be from about 3000 cP (3 Pa*s) to about 25,000 cP (25 Pa*s), alternatively from about 3500 cP (3.5 Pa*s) to about 20,000 cP (20 Pa*s), alternatively from about 4000 cP (4 Pa*s) to about 15,000 cP (15 Pa*s), or alternatively from about 5000 cP (5 Pa*s) to about 10,000 cP (10 Pa*s). The average viscosity can be determined by the Viscosity Test Method, described hereafter.

The composition can contain greater than or equal to 0.5%, alternatively greater than or equal to 0.6%, alternatively greater than or equal to 0.7%, alternatively greater than or equal to 0.8%, alternatively greater than or equal to 0.9%, or alternatively greater than or equal to 1.0% sodium hyaluronate. The composition can contain from about 0.5% to about 5%, alternatively from about 0.6% to about 4%, alternatively from about 0.75% to about 3%, alternatively from about 0.9% to about 2%, alternatively from about 1% to about 1.5% sodium hyaluronate.

The sodium hyaluronate can have a Mw of from about 200,000 Da to about 1.5 million Da, alternatively from about 300,000 Da to about 1.25 million Da, alternatively from about 400,000 Da to about 1 million Da, or alternatively from about 500,000 Da to about 950,000 Da. The sodium hyaluronate can have a Mw of less than 3 million Da, alternatively less than 2 million Da, alternatively less than 1 million Da. The sodium hyaluronate can have a Mw of greater than 100,000 Da, alternatively greater than 200,000 Da, alternatively greater than 300,000 Da, alternatively greater than 400,000 Da, alternatively greater than about 500,000 Da. The Mw of sodium hyaluronate can be determined according to the method described in the European Pharmacopoeia (European Pharmacopoeia 9.0. Sodium Hyaluronate. 01/2017) (hereinafter “European Pharmacopeia Method”).

The skin care composition can contain about 1% sodium hyaluronate with a Mw of from about 300,000 Da to about 1 million Da, alternatively from about 400,000 Da to about 950,000 Da, alternatively from about 500,000 Da to about 920,000 Da, alternatively from about 505,000 Da to about 855,000 Da, or alternatively from about 510,000 Da to about 620,000 Da and greater than 1.6% thickening agent, alternatively greater than 1.7% thickening agent, alternatively greater than 1.75% thickening agent, or alternatively greater than or equal to about 1.8% thickening agent.

The skin care composition can contain about 1% sodium hyaluronate with a Mw of from about 500,000 Da to about 1 million Da, alternatively from about 550,000 Da to about 950,000 Da, alternatively from about 575,000 Da to about 920,000 Da, alternatively from about 600,000 Da to about 855,000 Da, alternatively from about 610,000 Da to about 800,000, alternatively from about 615,000 Da to about 750,000 Da, or alternatively from about 615,000 Da to about 700,000 Da and greater than 1.5% thickening agent, alternatively greater than 1.55% thickening agent, or alternatively greater than or equal to 1.6% thickening agent.

The skin care composition can contain about 1% sodium hyaluronate with a Mw of from about 700,000 Da to about 2 million Da, alternatively from about 750,000 Da to about 1.5 million Da, alternatively from about 800,000 Da to about 1.25 million Da, alternatively from about 825,000 Da to about 1 million Da, alternatively from about 850,000 Da to about 950,000 Da, or alternatively from about 855,000 Da to about 920,000 Da and greater than 0.5% thickening agent, alternatively greater than 0.75% thickening agent, greater than 0.8% thickening agent, greater than 0.85% thickening agent, or alternatively greater than or equal to 0.9% thickening agent.

The skin care composition can be phthalate free, paraben free, dye-free, free of synthetic fragrance, and/or fragrance free.

### Thickening Agent

Any or a combination the three compositions may include one or more thickening agents. The compositions may comprise greater than 0.5%, alternatively greater than 0.7%, alternatively greater than 0.9%, alternatively greater than 1.0%, alternatively greater than 1.1%, or alternatively greater than or equal to 1.2% thickening agent. The compositions may comprise less than or equal to 2% thickening agent, alternatively less than or equal to 1.9% thickening agent, or alternatively less than or equal to 1.8% thickening agent. The compositions may comprise from about 1% to about 2%, alternatively from about 1.2% to about 2.5%, alternatively from about 1.3% to about 2.2%, alternatively from about 1.4% to about 2.0%, or alternatively from about 1.5% to about 1.8% of a thickening agent. Suitable classes of thickening agents include but are not limited to carboxylic acid polymers, polyacrylamide polymers, sulfonated polymers, copolymers thereof, hydrophobically modified derivatives thereof, and mixtures thereof. The thickening agent can be a polyacrylamide polymer and copolymer.

Suitable thickening agents include carboxylic acid polymers such as the carbomers (e.g., the CARBOPOL® 900 series such as CARBOPOL® 954), and Ultrez 10 and Ultrez 30. Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, Ultrez 20, Ultrez 21, PEMULEN TR-1, and PEMULEN TR-2, from Noveon, Inc.

Other suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic® Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.).

Other suitable thickening agents useful herein are sulfonated polymers such as the CTFA designated sodium polyacryloyldimethyl taurate available under the trade name Simulgel 800 from Seppic® Corp. and Viscolam® at 100 P available from Lambeth S.p.A. (Gallarate, Italy). Another commercially available material comprising a sulfonated polymer is Sepiplus™ 400 available from Seppic® Corp.

Further, suitable thickening agents may include superabsorbent polymers. These superabsorbent polymers may be chosen from: crosslinked sodium polyacrylates, such as, for example, those sold under the names Octacare X100, X110 and RM100 by Avecia®, those sold under the names Flocare GB300 and Flosorb 500 by SNF™, those sold under the names Luquasorb 1003, Luquasorb 1010, Luquasorb 1280 and Luquasorb 1100 by BASF®, those sold under the names Water Lock G400 and G430 (INCI name: Acrylamide/Sodium Acrylate Copolymer) by Grain Processing®, or Aqua Keep® 10 SH NF, Aqua Keep® 10 SH NFC, sodium acrylate crosspolymer-2, provided by Sumitomo Seika, starches grafted by an acrylic polymer (homopolymer or copolymer) and in particular by sodium polyacrylate, such as those sold under the names Sanfresh ST-100C, ST100MC and IM-300MC by Sanyo Chemical Industries®, Makimousse 12 and Makimouse 25 supplied by Kobo Products Inc (INCI name: Sodium Polyacrylate Starch), hydrolysed starches grafted by an acrylic polymer (homopolymer or copolymer), in particular the acryloacrylamide/sodium acrylate copolymer, such as those sold under the names Water Lock A-240, A-180, B-204, D-223, A-100, C-200 and D-223 by Grain Processing® (INCI name: Starch/Acrylamide/Sodium Acrylate Copolymer). Preferred superabsorbent polymers can include Makimousse 12 and Makimousse 25.

Suitable thickening agents for use herein include gums. “Gum” is a broadly defined term in the art. Gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, derivatives thereof and mixtures thereof.

Natural gums are polysaccharides of natural origin, capable of causing a large viscosity increase in solution, even at small concentrations. They can be used as thickening agents, gelling agents, emulsifying agents, and stabilizers. Most often these gums are found in the woody elements of plants or in seed coatings. Natural gums can be classified according to their origin. They can also be classified as uncharged or ionic polymers (polyelectrolytes), examples of which include the following. Natural gums obtained from seaweeds, such as: agar; alginic acid; sodium alginate; and carrageenan. Natural gums obtained from non-marine botanical resources include: gum arabic, from the sap of Acacia trees; gum ghatti, from the sap of Anogeissus trees; gum tragacanth, from the sap of Astragalus shrubs; karaya gum, from the sap of Sterculia trees. Examples of uncharged gums include: guar gum, from guar beans, locust bean gum, from the seeds of the carob tree; beta-glucan, from oat or barley bran; chicle gum, an older base for chewing gum obtained from the chicle tree; dammar gum, from the sap of Dipterocarpaceae trees; glucomannan from the konjac plant; mastic gum, a chewing gum from ancient Greece obtained from the mastic tree; psyllium seed husks, from the Plantago plant; spruce gum, a chewing gum of American Indians obtained from spruce trees; tara gum, from the seeds of the tara tree. Natural gums produced by bacterial fermentation include gellan gum and xanthan gum.

### Vitamin B₃ Compound

The compositions herein can include a safe and effective amount of a vitamin B₃ compound. In some instances, the present compositions may contain 0.01% to 10%, by weight, of the vitamin B₃ compound, based on the weight or volume of the composition (e.g., 0.1% to 10%, 0.5% to 5%, or even 1% to 3%).

As used herein, “vitamin B₃ compound” means a compound having the formula:

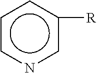

Where: R is CONH₂ (i.e., niacinamide), COOH (i.e., nicotinic acid) or CH₂OH (i.e., nicotinyl alcohol); derivatives thereof; and salts of any of the foregoing.

Exemplary derivatives of vitamin B₃ compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion that has a continuous aqueous phase (e.g., an oil-in-water or water-in-oil-in-water emulsion) or a continuous oil phase (e.g., water-in-oil or oil-in-water-in-oil emulsion). The oil phase of the emulsion may include silicone oils, non-silicone oils such as hydrocarbon oils, esters, ethers, and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). The composition can contain from about 1% to about 15%, alternatively from about 3% to about 10%, alternatively from about 4% to about 9%, and alternatively from about 5% to about 8% humectant.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., molecular weight of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Silicone Oil

The composition can include a silicone oil selected from volatile silicone oil, non-volatile silicone oil, and combinations thereof. The silicone oil can be in the dispersed phase. The composition can include from about 1% to about 10% silicone oil, alternatively from about 3% to about 8% silicone oil, and alternatively from about 4% to about 7% silicone oil.

### Volatile Silicone Oil

Suitable volatile silicones include cyclic and linear volatile silicones. A description of various volatile silicones is found in Todd, et al. “Volatile Silicone Fluids for Cosmetics”, 91 Cosmetics and Toiletries 27-32 (1976). Suitable cyclic volatile silicones include cyclic dimethyl siloxane chains containing an average of from about 3 to about 5 silicon atoms, preferably from about 4 to about 5 silicon atoms. Exemplary cyclic volatile silicones of varying viscosities include Dow Corning DC 244, DC 245, DC 344, and DC 345; GE Silicones-OSi Specialties Volatile Silicone 7207 and Volatile Silicone 7158; and GE Silicones SF1202. Suitable volatile linear silicones include the polydimethylsiloxanes containing an average of from about 2 to about 8 silicon atoms. Exemplary linear volatile silicones include the Dow Corning DC 200 series with viscosities of 0.65 cst, 1.0 cst, and 2.0 cst. In certain embodiments, the linear volatile silicones generally have viscosities of less than or equal to about 4 centistokes at 25° C., and the cyclic materials generally have viscosities of less than about 6 centistokes at 25° C.

### Non-Volatile Silicone Oils

Suitable non-volatile silicone oils include polysiloxanes. Non-volatile polylsiloxanes may have a viscosity of from about 10 to about 1,000,000 centistokes at 25° C. Such polysiloxanes can be represented by the general chemical formula:

R3SiO[R2SiO]xSiR3

wherein each R is independently selected from hydrogen or C1-30 straight or branched chain, saturated or unsaturated alkyl, phenyl or aryl, trialkylsiloxy; and x is an integer from 0 to about 10,000. In certain embodiments, R is methyl or ethyl. Commercially available polysiloxanes include the polydimethylsiloxanes, which are also known as dimethicones, examples of which include the DM-Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids). Suitable dimethicones include those represented by the chemical formula:

R3SiO[R2SiO]x[RR′SiO]ySiR3

wherein R and R′ are each independently hydrogen or C1-30 straight or branched chain, saturated or unsaturated alkyl, aryl, or trialkylsiloxy; and x and y are each integers of 1 to 1,000,000. Examples include alkyl dimethicones wherein at least R′ is a fatty alkyl (e.g., C12-22). A suitable alkyl dimethicone is cetyl dimethicone, wherein R′ is a straight C16 chain and R is methyl, commercially available as 2502Cosmetic Fluid from Dow Corning.

Preferred non-volatile oils include dimethicones (polydimethylsiloxanes), preferably with viscosities of between 10 cst and 1000 cst, more preferably between 15 cst to 400 cst, most preferably between 20 cst and 200 cst. The average chain lengths for these preferred dimethicone materials is from about 12 to about 375 dimethylsiloxane units, more preferably from about 20 to about 200 dimethylsiloxane units, and most preferably with average chain lengths of from about 27 to about 125 dimethylsiloxane units. In one embodiment, the second composition will comprise at least one non-volatile silicone oil. In one such embodiment, at least about 70%, by weight of the non-volatile oil, is a non-volatile silicone oil. In another embodiment, at least about 80%, by weight of the nonvolatile oil, is a non-volatile silicone. In yet another embodiment, at least about 90%, by weight of the non-volatile oil, is a non-volatile silicone oil.

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. In some examples, the composition can include a fragrance, in particular a natural fragrance, or a colorant, in particular a natural colorant. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use

The skin care serum composition may be applied to the face, neck, and/or a portion or combination thereof at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period herein is ideally of sufficient time for the sodium hyaluronate and/or other skin care actives to improve the appearance of the skin. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The serum composition can be applied to a dry face after cleansing. The serum composition can be applied before or after other skin care compositions including but not limited to moisturizer and/or sunscreen.

The serum composition can be intended to help improve the appearance of fine lines and wrinkles. The serum composition can be stronger and last longer, up to two times longer than other commercially available products. The serum can leave skin dewy and hydrated.

### Examples

The Examples in Table 4, below, could be prepared by conventional methods of making topical skin care emulsions. The following examples could be made by combining the water phase ingredients (e.g., sodium hyaluronate, glycerin, skin care actives, chelants, actioxidants, preservatives, and emulsifiers) in a container and mixing until uniform. The oil phase ingredients (e.g., oils including silicone oils and emulsifiers) can be combined in a separate container and mixed until uniform. The oil phase can then be added to the water phase and the resulting emulsion can be subjected to high shear mixing (e.g., Flacktek Speedmixer®, or rotor-stator mill). Finally, the remaining thickener(s) can be added to the emulsion and mixed until uniform, and then the pH adjuster, if present, is added and the product is subjected to high shear mixing until uniform.

TABLE 4
Serum Examples

| | Ex. 1 | Ex. 2 | Ex. 3 | Ex.4 | Ex. 5 | Ex. 6 | Ex. 7 |
|---|---|---|---|---|---|---|---|
| Water Phase | | | | | | | |
| Water | QS | QS | QS | QS | QS | QS | QS |
| Sodium Hyaluronate | 0.5 | 1.5 | 1.0 | 0.75 | 1.0 | 1.0 | 0.5 |
| Dex-Panthenol | 0.5 | 0.5 | 0.5 | — | — | — | 0.5 |
| Glycerin | 7 | 10 | — | 3 | — | 7 | 15 |
| Butylene Glycol | — | — | 4 | — | — | — | — |
| Propylene Glycol | — | — | — | — | 6 | — | — |
| Disodium EDTA | 0.05 | 0.025 | 0.05 | 0.025 | 0.025 | — | 0.05 |
| Hydroxyacetophenone | 0.15 | 0.15 | 0.05 | — | 0.15 | — | 0.2 |
| Ethylhexylglycerine | 0.2 | 0.1 | — | — | — | — | — |
| Phenoxyethanol | 0.05 | 0.25 | 0.5 | — | 0.15 | — | 0.2 |
| Symdiol 68 ®¹ | — | — | — | 0.4 | — | — | — |
| Glycacil ® L² | — | — | — | 0.09 | — | — | — |
| Glydant Plus ® Liquid³ | — | — | — | — | — | 0.3 | — |
| Niacinamide | 2.0 | 2.0 | 5.0 | 3.5 | — | 2.0 | 3.0 |
| Sepiwhite ® MSH⁴ | — | — | — | — | 1.0 | — | — |
| Glyco-Repair ™⁵ | — | — | 2.0 | — | 1.0 | — | — |
| Palestrina ®⁶ | 0.6 | — | 0.4 | — | 1.0 | — | — |
| Inositol | — | 0.5 | — | — | — | — | — |
| Olivem ® 460⁷ | 0.1 | — | — | 0.1 | — | — | — |
| Aloe Vera Gel | — | — | 0.5 | — | — | — | — |
| Promatrixyl ®⁸ | 0.4 | — | 0.7 | 0.05 | — | — | 0.3 |
| Green Tea Extract | — | — | — | — | — | 1.0 | 1.0 |
| pH Adjustor | | | | | | | |
| Triethanolamine | 0.45 | — | — | — | 0.2 | — | — |
| Aminomethyl | — | — | — | 0.1 | — | 0.1 | — |
| Propanol | | | | | | | |
| Thickener | | | | | | | |
| Sepigel ™ 305⁹ | 0.9 | 1.8 | 2.0 | — | 0.4 | 1.5 | — |
| Simulgel ® INS-100¹⁰ | — | — | — | 0.9 | — | — | — |
| Makimousse-12¹¹ | — | — | — | — | — | — | 0.5 |
| Ultrez-10¹² | — | — | — | 0.1 | — | — | — |
| Ultrez-21¹³ | — | — | — | 0.1 | — | — | — |
| Xanthan Gum | — | — | — | — | — | — | 0.1 |
| Oil Phase | | | | | | | |
| Cyclomethicone D5 | 4.0 | 1.0 | 2.0 | — | — | — | — |
| Dimethicone 2 cst | 2.0 | — | 2.0 | — | — | — | — |
| Dimethicone 5 cst | | 5.0 | | 3.0 | | 4.0 | 2.0 |
| Dimethicone 50 cst | | | | 2.0 | 3.0 | 2.0 | — |
| Dimethicone 350 cst | — | — | 1.0 | — | — | — | — |
| DC9041¹⁴ | 0.5 | — | — | 1.0 | — | — | 2.0 |
| Hexyldecanol | — | — | — | 0.1 | 5.0 | — | — |
| Retinyl Propionate | 0.3 | — | — | 0.3 | — | — | — |
| Laureth-4 | 0.1 | 0.2 | 0.3 | — | 0.2 | 0.1 | 0.3 |
| DC1503¹⁵ | — | 2.0 | — | — | 1.5 | — | — |
| Polysorbate 20 | 0.1 | 0.2 | — | 0.3 | 0.2 | 0.2 | ___ |

¹1,2-hexanediol and caprylyl glycol, from Symrise ®
²Iodopropynyl butylcarbamate, PEG-4 laurate, PEG-4 dilaurate, and polyethylene glycol, from Lonza ®
³DMDM Hydantoin, Butane-1,3-diol, iodopropynyl butylcarbamate, water, from Lonza ®
⁴Undecylenoyl phenylalanine, from Seppic ® Corporation
⁵Water and hydrolyzed ceratonia siliqua seed extract, from Silab ®
⁶Water, glycerin, decyl glucoside, lactic acid, benzyl alcohol, and palmitoyl dipeptide-7, from Sederma ®
⁷Sodium PEG-7 olive oil carboxylate, from B&T S.r.l.
⁸Water, glycerin, PEG-100 stearate, benzyl alcohol, and palmitoyl pentapeptide-4, from Sederma ®
⁹Polyacrylamide, C13-14 Isoparaffin, and laureth-7, from Seppic ® Corporation
¹⁰Sodium polyacrylate starch, from Kobo ® Products Inc.
¹²Carbomer, from Lubrizol ®
¹³Acrylates C10-30 alkyl acrylate crosspolymer, from Lubrizol ®
¹⁴Dimethicone (and) dimethicone crosspolymer, from Dow Corning ®
¹⁵Dimethicone (and) dimethiconol, from Dow Corning ®

### Test Methods

### Viscosity Test Method

After storing for at least 24 hours at 25° C., the product viscosity is measured by a DV2T-RV viscometer (available from Brookfield Engineering Laboratories, Middleboro, Mass.) with a TC spindle rotated at 5 rpm at 25° C. The spindle starts 2.5-3.0 cm below the product surface and travels a helipath downward and collects one reading every 9 seconds for 5 readings. The average (mean) of the readings is calculated to determine the average viscosity.

### Combinations

A. A physically stable skin care serum composition comprising:
   a. a continuous phase comprising:
      i. from about 0.5% to about 3% sodium hyaluronate comprising a weight average molecular weight from about 500,000 to about 1,000,000 Da, preferably from about 500,000 Da to about 920,000 Da, more preferably from about 505,000 Da to about 855,000 Da, and even more preferably from about 510,000 Da to about 620,000, according to the European Pharmacopoeia Method.
      ii. greater than 0.7% thickening agent, preferably greater than 1.0% thickening agent, more preferably greater than 1.1% thickening agent, and even more preferably greater than or equal to 1.2% thickening agent; wherein the thickening agent is a polyacrylamide polymers and copolymers;
      iii. water;
   b. a dispersed phase comprising silicone;
      wherein the composition comprises a viscosity from about 3,000 cP to 25,000 cP.
B. A physically stable skin care serum composition comprising:
   a. from about 0.5% to about 3% sodium hyaluronate comprising a weight average molecular weight from about 700,000 Da to about 2 million Da, preferably from about 750,000 Da to about 1.5 million Da, more preferably from about 800,000 Da to about 1.25 million Da, and even more preferably from about 855,000 Da to about 920,000 Da, according to the European Pharmacopoeia Method;
   b. greater than 1% thickening agent, more preferably greater than 1.1% thickening agent, and even more preferably greater than or equal to 1.2% thickening agent;
   wherein the thickening agent is polyacrylamide polymers and copolymers;
   c. water;
      wherein the composition comprises an average viscosity from about 3,000 cP (3 Pa*s) to 25,000 cP (25 Pa*s).
C. The composition of Paragraph B, wherein the skin care composition is an emulsion comprising a continuous phase and a dispersed phase; wherein the continuous phase comprises the sodium hyaluronate, the thickening agent, and the water, and wherein the dispersed phase comprises silicone.
D. The composition according to Paragraphs A-C, wherein the average viscosity is from about 3000 cP (3 Pa*s) to about 25,000 cP (25 Pa*s), preferably from about 3500 cP (3.5 Pa*s) to about 20,000 cP (20 Pa*s), more preferably from about 4000 cP (4 Pa*s) to about 15,000 cP (15 Pa*s), and even more preferably alternatively from about 5000 cP (5 Pa*s) to about 10,000 cP (10 Pa*s) according to the Viscosity Test Method, described herein.
E. The composition according to Paragraphs A-D, wherein the composition comprises from about 0.6% to about 2% sodium hyaluronate, preferably from about 0.7% to about 1.5%, and more preferably from about 0.75% to about 1.25% sodium hyaluronate.
F. The composition according to Paragraphs A-E, wherein the composition comprises less than or equal to 2% thickening agent, preferably less than or equal to 1.9% thickening agent, and preferably less than or equal to 1.8% thickening agent.
G. The composition according to Paragraphs A-F, wherein the thickening agent comprises polyacrylamide and isoparaffin and laureth-7.
H. The composition according to Paragraphs A-G, wherein the composition is free of phthalate, paraben, dye, synthetic fragrance, and/or fragrance.
I. The composition according to Paragraphs A-H, wherein the continuous phase further comprises from about 3% to about 10% glycerin, preferably from about 4% to about 9% glycerin, and more preferably from about 5% to about 8% glycerin.
J. The composition according to Paragraphs A-I, wherein the continuous phase further comprises a vitamin B₃ compound selected from niacinamide, nicotinic acid, nicotinyl alcohol, and combinations thereof.
K. A skin care product comprising a dropper package adapted for storing and dispensing the skin care serum composition of Paragraphs A-J.
L. Use of the skin care serum composition of Paragraphs A-K to increase skin hydration and/or reduce the appearance of fine lines or wrinkles.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A physically stable skin care serum composition comprising:
a. a continuous phase comprising:
i. from about 0.5% to about 1.5% sodium hyaluronate comprising a weight average molecular weight from about 500,000 to about 1,000,000 Da;
ii. greater than 0.7% thickening agent; wherein the thickening agent is a polyacrylamide polymer and copolymer;
iii. water;
b. a dispersed phase comprising silicone;
wherein the composition comprises an average viscosity of from about 4,000 cP to 25,000 cP.

2. The composition of claim 1, wherein the composition comprises from about 1% to about 2% thickening agent.

3. The composition of claim 1, wherein the thickening agent comprises polyacrylamide and isoparaffin and laureth-7.

4. The composition of claim 1, wherein the composition is free of phthalate, paraben, dye, synthetic fragrance, and/or fragrance.

5. The composition of claim 1, wherein the continuous further comprises from about 3% to about 10% glycerin.

6. The composition of claim 1, wherein the continuous phase further comprises a vitamin B₃ compound selected from niacinamide, nicotinic acid, nicotinyl alcohol, and combinations thereof.

7. The composition of claim 1, wherein the average viscosity is from about 5000 cP to about 10,000 cP.

8. The composition of claim 1, wherein the sodium hyaluronate comprises a weight average molecular weight from about 700,000 to about 1,000,000 Da.

9. A skin care product comprising a dropper package adapted for storing and dispensing the skin care serum composition of claim 1.

10. A physically stable skin care serum composition comprising:
a. from about 0.5% to about 1.5% sodium hyaluronate comprising a weight average molecular weight from about 700,000 to about 1,000,000 Da;
b. greater than 1% thickening agent; wherein the thickening agent is a polyacrylamide polymer and copolymer;
c. water;
wherein the composition comprises an average viscosity of from about 4,000 cP to 25,000 cP.

11. The composition of claim 0, wherein the composition comprises greater than 1.2% thickening agent.

12. The composition of claim 0, wherein the thickening agent comprises polyacrylamide and isoparaffin and laureth-7.

13. The composition of claim 0, wherein the skin care composition is an emulsion comprising a continuous phase and a dispersed phase; wherein the continuous phase comprises the sodium hyaluronate, the thickening agent, and the water; and wherein the dispersed phase comprises silicone.

14. The composition of claim 0, wherein the continuous phase further comprises from about 3% to about 10% glycerin.

15. The composition of claim 0, wherein the continuous phase further comprises a vitamin B₃ compound selected from niacinamide, nicotinic acid, nicotinyl alcohol, and combinations thereof.

16. The composition of claim 0, wherein the average viscosity is from about 5000 cP to about 10,000 cP.

17. The composition of claim 0, wherein the sodium hyaluronate comprises a weight average molecular weight from about 850,000 Da to about 950,000 Da.

18. The composition of claim 0, wherein the composition is free of phthalate, paraben, dye, synthetic fragrance, and/or fragrance.

19. A skin care product comprising a dropper package adapted for storing and dispensing the skin care serum composition of claim 0.
